(19) United States
(12) Patent Application Publication
(10) Pub. No.: US 2012/0078087 A1
(43) Pub. Date: Mar. 29, 2012

(54) Tissue Localization Device and Method

(76) Inventor: Kyle S. Curry, Carmel, IN (US)

(21) Appl. No.: 12/888,801

(22) Filed: Sep. 23, 2010

(51) Int. Cl. A61B 10/02; A61B 5/055

(52) U.S. Cl. 600/424; 600/567

(57) ABSTRACT

A system for localizing tissue in a single MRI session is disclosed. The system comprises an MRI compatible localizing obturator selectively insertable through the working channel of an introducer cannula or sheath and configured for insertion through a tissue pathway to a target tissue site. The obturator includes an elongate shaft having open proximal and distal ends and an inner lumen extending between the open proximal and distal ends. At least one MRI image-enhancing component is disposed on and/or within the distal end of the obturator. The system further includes an MRI compatible localizing wire slidable within the inner lumen of the obturator. The wire defines an elongate body having first and second ends and an anchoring member adjacent to one of the first and second ends, the anchoring member adapted to engage tissue so that the wire remains in the patient upon removal of the obturator.

### FIELD OF THE INVENTION

The present invention relates to a system and method useful in localizing a target tissue site, such as breast biopsy site and, more particularly, to a system and method that may be useful in localizing a target tissue site under the guidance of magnetic resonance imaging (MRI) using a localizing obturator in conjunction with a localizing wire.

### BACKGROUND OF THE INVENTION

Tissue biopsy using a biopsy needle or cannula is a commonly used procedure for diagnosing the presence of a malignancy in a suspicious tissue mass comprising an anomaly, such as a lesion. However, some women are not candidates for this type of biopsy for technical reasons, such as, for example, having breasts that are too small for the biopsy probe, or if the lesion is either very superficial or very deep. In these instances, a surgical biopsy, such as wire localization biopsy, is the accepted medical approach to evaluate and, if necessary, treat the suspected malignancy at the earliest possible time.

Upon completing a pre-surgery evaluation, the suspicious tissue mass is localized during a localization procedure using an imaging technique such as radiography, ultrasound, or magnetic resonance imaging (MRI), and a clip or marker is inserted into the tissue mass through a surgically inserted introducer sheath. The marker serves as an imageable reference for locating the tissue mass during subsequent evaluations and procedures.

While the marker can locate the lesion through imaging techniques, it is inadequate as a guide for the biopsy surgeon to quickly locate the lesion with minimal trauma to the surrounding tissue. Consequently, after the lesion is localized during the initial localization procedure, the patient must return to the hospital or doctor's office to undergo a subsequent wire localization procedure. During this procedure, a localizing wire is inserted into the patient and secured to the tissue mass in the area of the lesion, serving as a tangible means to guide the surgeon directly to the lesion during a biopsy or lumpectomy. This can be an extremely painful procedure for the patient. The procedure is also time-consuming and costly. Additionally, since the marker is typically used to relocate the lesion during placement of the localizing wire, it is possible to mislocate the localizing wire, particularly if the marker has migrated. In such a case, the lesion may not be sufficiently excised, or excess, healthy tissue may be unnecessarily excised.

### SUMMARY

According to one aspect, the present invention is directed to a system for localizing a tissue mass. The system comprises an introducer cannula, which is selectively insertable into a patient's tissue and includes a working channel extending therethrough; and an MRI compatible localizing obturator, which is selectively insertable within the working channel of the introducer cannula and configured for insertion through a tissue pathway leading to a target tissue site. The obturator includes an elongate shaft having an open proximal end, an open a distal end and an inner lumen extending between the open proximal and distal ends. At least one image enhancing component visible under magnetic resonance imaging (MRI) is disposed on and/or within at least a portion of the obturator. The system further comprises an MRI compatible localizing wire slidable within the inner lumen of the obturator. The localizing wire includes an elongate body having first and second ends and an anchoring member formed on one of the first and second ends, which is adapted to engage tissue.

In one embodiment, the system further comprises an introducer stylet selectively insertable within the working channel of the introducer cannula. The stylet includes a tissue piercing tip and is configured to create a pathway to a target tissue site through which at least one of the introducer cannula and obturator is inserted.

In some embodiments, the distal end of the obturator defines a blunt tip or a tissue-piercing tip; and in some such embodiments, the tissue piercing tip is a selectively removable tissue piercing tip attached to the distal end of the obturator.

Further, in some embodiments, the localizing obturator and localizing wire are visible under multiple imaging modalities. Still further, in some embodiments the obturator further comprises a port at its proximal end, which is adapted to connect to a fluid and/or vacuum source to introduce fluid and/or vacuum through the inner lumen.

In some embodiments, the distal end of the obturator defines an outer surface including at least one MRI image enhancing component and/or at least one inner chamber including at least one MRI image enhancing component. And in some such embodiments, the distal end of the obturator defines an outer surface and at least one inner chamber between the outer surface and the inner lumen, and the at least one image enhancing component is disposed on the outer surface and/or within the at least one inner chamber.

In some embodiments, the image-enhancing component is at least one of a metallic material and a liquid contrast agent. Further, in some embodiments at least one of the localizing obturator and localizing wire is formed from at least one of 316 stainless steel, Inconel 625, a ceramic, glass, titanium and a polymer.

Still further, in some embodiments, the system further comprises a marker deployment device insertable within the working channel of the introducer cannula and adapted to introduce at least one image marker into a target tissue site. Further, in some embodiments the system further comprises a structure adapted to support and position the introducer cannula relative to a target tissue site.

According to another aspect, the invention is directed to medical procedure. The procedure comprises the step of inserting an MRI compatible localizing obturator through a tissue pathway and into a target tissue site. The obturator includes an elongate shaft having an open proximal end, an open distal end and an inner lumen extending between the proximal and distal ends, wherein at least a portion of the obturator is visible by MRI. The procedure further comprises the steps of: positioning the obturator and localizing the target tissue site under MRI guidance; selectively inserting an MRI compatible localizing wire through the inner lumen of the obturator and into the target tissue site, wherein at least a portion of the localizing wire is visible by MRI; and positioning the localizing wire under MRI guidance so that a portion of the wire is placed at the target tissue site. Still further, the procedure comprises the step of removing the localizing obturator from the target tissue site and tissue pathway, while leaving behind the localizing wire such that a distal portion of the localizing wire remains in contact with the target tissue site and a proximal portion of the wire extends completely through the tissue pathway. In some embodiments, the entire procedure is performed in a single MRI session.

In some embodiments, the localizing wire includes an elongate body having first and second ends and an anchoring member formed on one of the first and second ends, the anchoring member adapted to engage tissue. Accordingly, the step of inserting the localizing wire further comprises the step of positioning the anchoring member to engage tissue within or adjacent to the target site to secure the localizing wire to tissue.

Further, in some embodiments, the procedure further comprises the step of providing a structure adapted to support and aid in positioning the obturator relative to a target tissue site.

According to another aspect, the invention is directed to a method for localizing a breast biopsy site. The method comprises the steps of: inserting an introducer stylet through the working channel of an introducer cannula; inserting the stylet and introducer cannula into a patient's breast tissue, creating a pathway to a target tissue site; and inserting an MRI compatible localizing obturator into the working channel of the introducer cannula and through the tissue pathway to the target tissue site. The obturator includes an elongate shaft having an open proximal end, an open distal end and an inner lumen extending between the proximal and distal, wherein at least a portion of the obturator is visible by MRI.

The method further comprises the steps of: positioning the obturator and localizing the target tissue site under MRI guidance and selectively inserting an MRI compatible localizing wire through the inner lumen of the obturator and into the target tissue site. The localizing wire includes an elongated body member having first and second ends and an anchoring member formed on one of the first and second ends and adapted to engage tissue. Still further, the method comprises the steps of positioning the localizing wire under MRI guidance so that the anchoring member engages tissue at or adjacent to the target tissue site to maintain the wire in contact with the target tissue site; and removing the localizing obturator from the patient while leaving behind the localizing wire such that a distal portion of the localizing wire remains in contact with the target tissue site and a proximal portion of the wire extends through the tissue pathway and outside of the patient. In some embodiments, the entire method is performed in single MRI session.

In some embodiments, the method further comprises the step of providing a structure adapted to support and position the introducer cannula relative to a target tissue site. And in some embodiments, the method further comprises the steps of selectively inserting a marker deployment device into the working channel of the introducer cannula and delivering a site marker to the target site.

Details of one or more implementations of the invention are set forth in the accompanying drawings and in the description below. Further features, aspects, and advantages of the invention will become apparent from the description, the drawings, and the claims.

### BRIEF DESCRIPTION OF DRAWINGS

FIG. 1 is a perspective view of a localizing obturator according to an embodiment of the invention.

FIG. 2 is a cross-sectional view of the obturator of FIG. 1.

FIG. 3 is a cross-sectional view of an embodiment of the distal end of the obturator of FIG. 1.

FIG. 4 is a side elevational view of an embodiment of a localizing wire usable in conjunction with the obturator of FIG. 1.

FIG. 5 is a cross-sectional view of the obturator of FIG. 1 with the localizing wire of FIG. 4 inserted therein.

FIG. 6A is a perspective view of an embodiment of the obturator of FIG. 1 having an integral tissue-piercing tip.

FIG. 6B is a perspective view of an embodiment of the obturator of FIG. 1 having a removable tissue-piercing tip.

FIG. 7 is a flow diagram of a method of using the obturator of in FIG. 1 in conjunction with the localizing wire of in FIG. 4 to localize and mark a target tissue site.

FIGS. 8-15 are elevational views illustrating a medical procedure using the obturator of FIG. 1 in conjunction with the localizing wire of FIG. 4 to localize and mark a breast biopsy site.

FIGS. 16A-16B are elevational views illustrating a medical procedure using the obturator of FIG. 1 in conjunction with the localizing wire of FIG. 4 to localize and mark a breast biopsy site.

Although the drawings represent embodiments of the present invention, the drawings are not necessarily to scale and certain features may be exaggerated in order to better illustrate and explain the present invention. The exemplification set out herein illustrates certain embodiments of the invention, in one, or more forms, and such exemplifications are not to be construed as limiting the scope of the invention in any manner.

### DETAILED DESCRIPTION

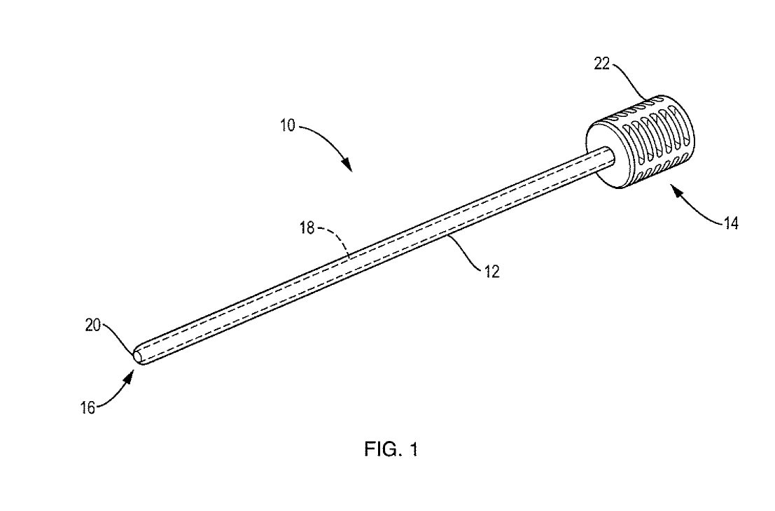

Referring to FIG. 1 a target localizing obturator embodying the present invention is indicated generally by the reference numeral 10. In at least one embodiment, the obturator 10 is used for assisting a physician in localizing (i.e. confirming the location of) a target tissue site, such as a biopsy site, for tissue marking purposes and is compatible with, and visible by, magnetic resonance imaging (MRI). Additionally, in cases where wire localization is deemed required for future evaluation and/or surgical treatment, the obturator 10 can be further be used as a wire localization guide and/or introducer, enabling the physician to insert a localizing wire through the obturator 10 to create a tangible and visible pathway under MRI guidance to the localized target site. Because the obturator 10 is MRI compatible and is adapted to receive an MRI compatible localizing wire, the physician can perform the wire localization procedure in the same MRI session where the target site is localized; thus advantageously obviating the need to perform the wire localization as a separate procedure at a different location outside of the MRI suite (and typically at a future time and date) using a different imaging modality, which is the current treatment method. Accordingly, medical expenses are reduced and, more importantly, the overall stress and time expenditure borne by the patient is minimized.

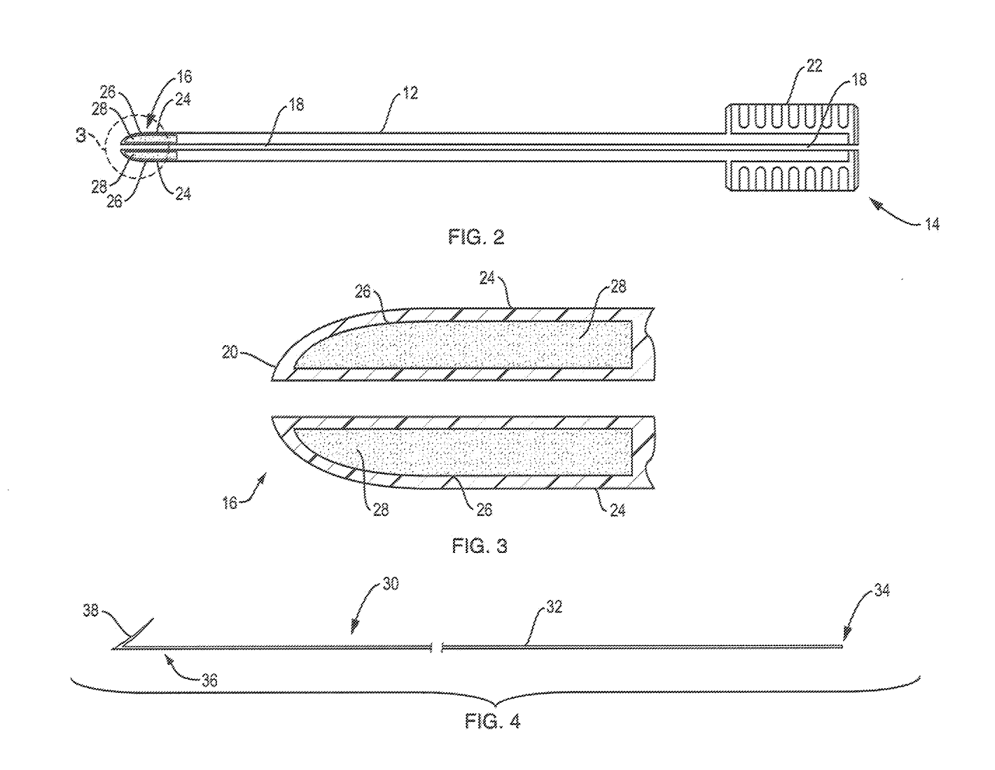

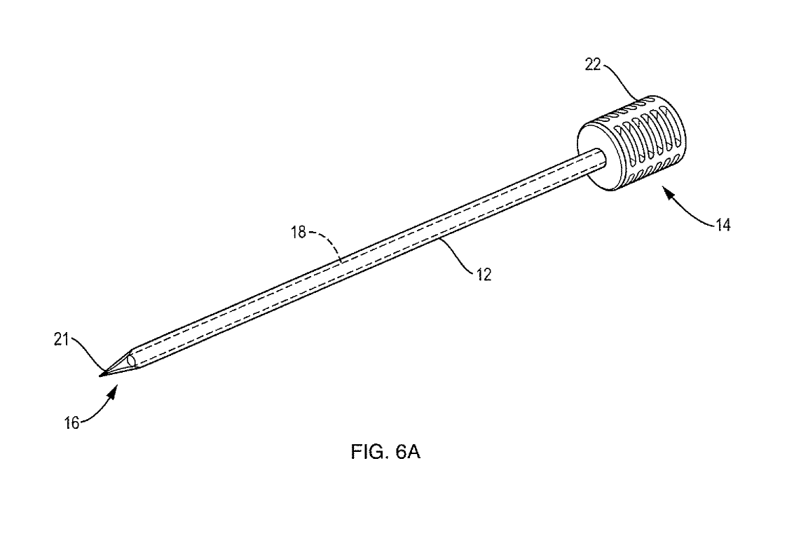

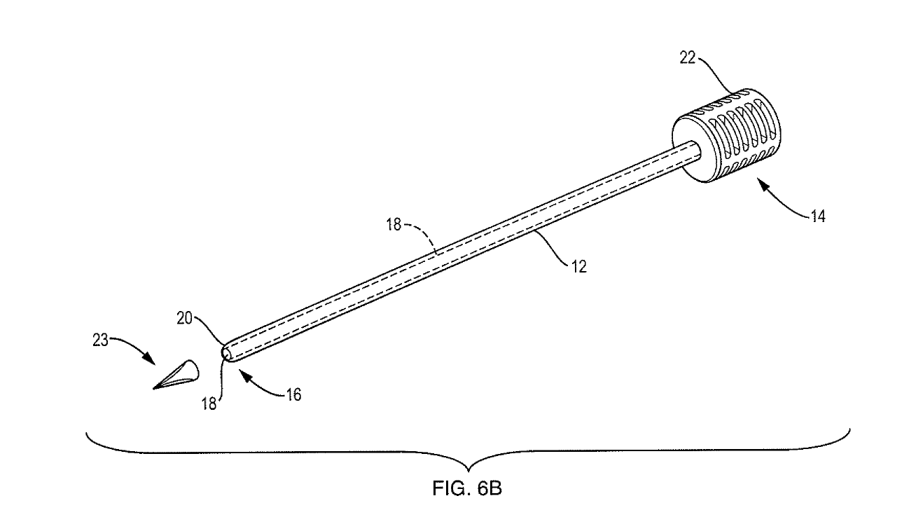

With reference to FIGS. 1-2, the obturator 10 comprises an elongate body or shaft 12 defining an open proximal end 14, an open distal end 16 and a lumen 18 extending between the open proximal and distal ends. In a preferred embodiment, the distal end defines a blunt tip 20 configured to penetrate through a pre-formed tissue pathway leading to a target site without piercing and/or cutting tissue. Further, the blunt tip 20 prevents unwanted tissue piercing and/or cutting when the obturator 10 is manipulated to localize the target tissue mass. However, if desired, the obturator 10 could define a tissue-piercing tip 21, such as the trocar tip shown in FIG.6A, allowing the obturator to further function as an introducer stylet capable of creating a tissue path to a target tissue site. Still further, the obturator 10 could include a selectively removable tissue-piercing tip 23 attached to its distal end 16 as shown, for example, in FIG. 6B. In this embodiment, with the tissue-piercing tip 23 attached, the obturator functions as a tissue-piercing stylet, and with the tissue-piecing tip removed, the obturator functions as the blunt localizing obturator shown in FIG. 1; hence, a separate tissue-piercing stylet is not required during the localization procedure. The distal end 16 of the obturator 10 further provides image enhancing and marking features, which are described in further detail below; however, it should be noted that any imaging enhancing features described herein could be applied to any portion of the obturator 10 in addition to or in place of the distal end 16.

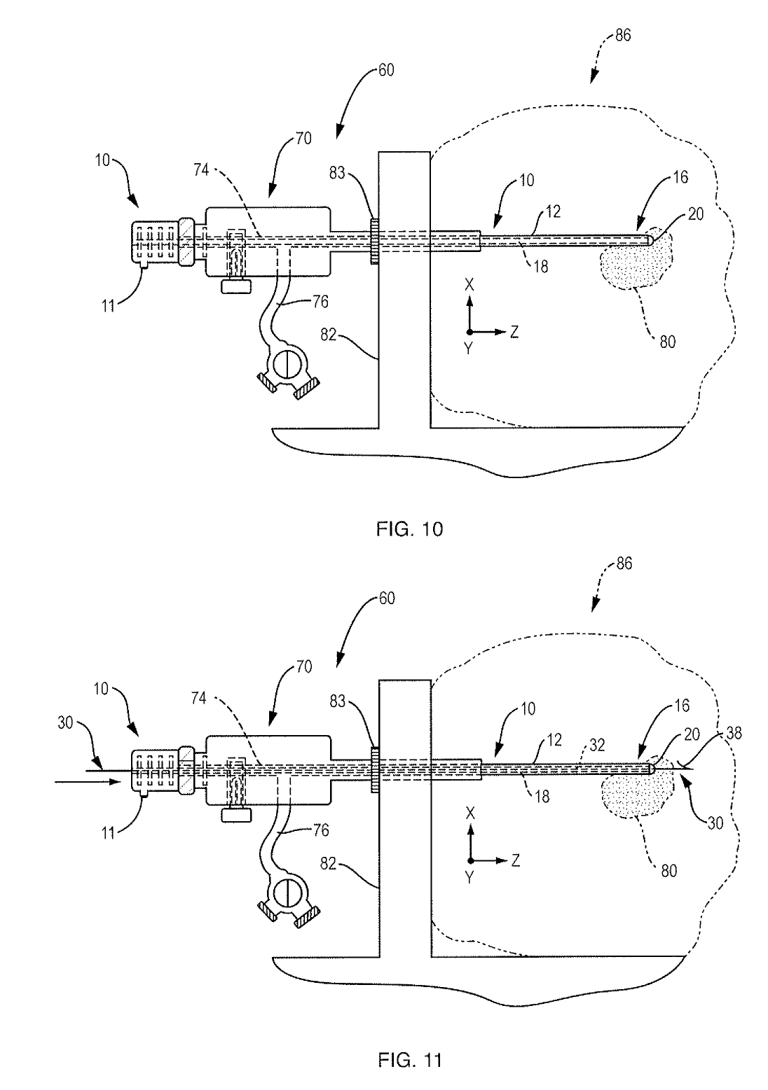

Opposite the distal end 16, a handle or gripping member 22 is disposed about the proximal end 14 of the shaft 12. The gripping member 22 can integrally form part of the proximal end 14 of the shaft 12 or can be a separately attachable member. The gripping member 22 is manually engageable and adapted to allow the physician to manipulate and position the obturator 10 during a target localization procedure. In one embodiment, shown for example in FIGS. 10-11, the obturator 10 further includes an optional fluid port 11 at its proximal end 14. The fluid port 11 is adapted to connect to a fluid and/or vacuum source to introduce fluid and/or vacuum through the inner lumen 18 to lavage and/or aspirate the target site during localization.

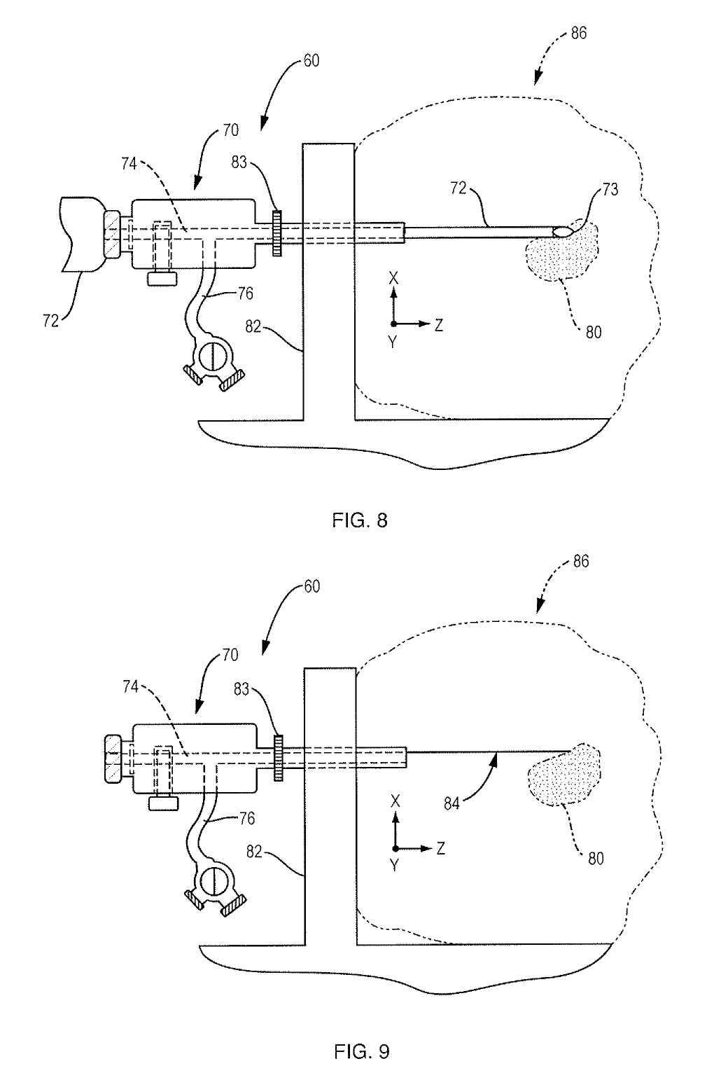

In the illustrated embodiment, the obturator shaft 12 has a generally cylindrical shape with an outer surface sized to fit within (and extend through) the inner lumen or working channel of an introducer sheath or cannula (for example, the inner lumen 74 of the introducer cannula 70 shown in FIG. 8). However, as recognized by those having ordinary skill in the art, the shaft 12 can take on any of numerous shapes and, therefore, the shape of the cannula should not be construed in a limiting sense. In some embodiments, the shaft 12 is made of a medical grade resin, a polymeric material such as polycarbonate or other MRI compatible material. However, for use with other imaging modalities, the shaft 12 could be made of any of numerous materials that are known to be compatible with the desired imaging modality as recognized by those having ordinary skill in the art.

Referring now to FIG. 3, an embodiment of the distal end 16 of the obturator 10 is shown in further detail. As illustrated, the distal end 16 further defines an outer surface 24 and at least one inner chamber 26. At least one image enhancing component 28 is disposed on the outer surface 24 of the distal end and/or within the at least one inner chamber 26; however, the image enhancing component 28 could be disposed on any portion of the obturator or even impregnated into the material(s) used to form the obturator itself. Hence, depending on where the image enhancing component is ultimately disposed, the distal end 16 may be configured without the at least one inner chamber 26 if desired. Further, although two chambers are shown in the illustrated embodiment, it should be noted that a single chamber or more than two chambers could be integrated therein. Still further, in some embodiments the distal end of the obturator defines an outer surface including at least one MRI image enhancing component and/or at least one inner chamber including at least one MRI image enhancing component. And in some such embodiments, the distal end of the obturator defines an outer surface and at least one inner chamber between the outer surface and the obturator lumen, and the at least one image enhancing component is disposed on the outer surface and/or within the at least one inner chamber.

In an exemplary embodiment, the image enhancing component 28 is preferably an MRI compatible and identifiable metallic material, such as Inconel® 625, titanium or other material with similar magnetic characteristics. Alternatively, a liquid contrast agent may be utilized. Suitable liquid contrast agents include polypropylene glycol (PPG), fluro-deoxyglucose (FDG), technicium 99, Gadolinium, and other MRI compatible contrast agents that are currently known or later become known. With the inclusion of the image enhancing component, the distal end 16 provides a visible reference point during MR imaging (or other suitable imaging modality) relative to the target tissue site of interest, such as a breast biopsy site, further aiding in localizing the site to determine the precise location of any suspicious tissue mass.

In addition to the MRI image enhancing components described above, alternative image enhancing components or materials known to those skilled in the art can be used, which are adapted to improve the visibility of the obturator 10 under one or more additional imaging modalities, including but not limited to, x-ray, ultrasound, tomography and nuclear medicine. Examples of suitable materials include, but are not limited to, titanium, stainless steel, ceramic, carbon, nickel titanium, platinum and glass. It is further conceivable that the image enhancing component can be formed in a predetermined identifiable shape or, alternatively, fills a cavity in the obturator 10 having a predetermined identifiable shape to further enhance visibility and provide assistance in distinguishing the obturator from its surroundings during imaging. For example, the image enhancing component cold be shaped like a ribbon, bow-tie or the Venus symbol or could fill a cavity in the obturator having one of these shapes.

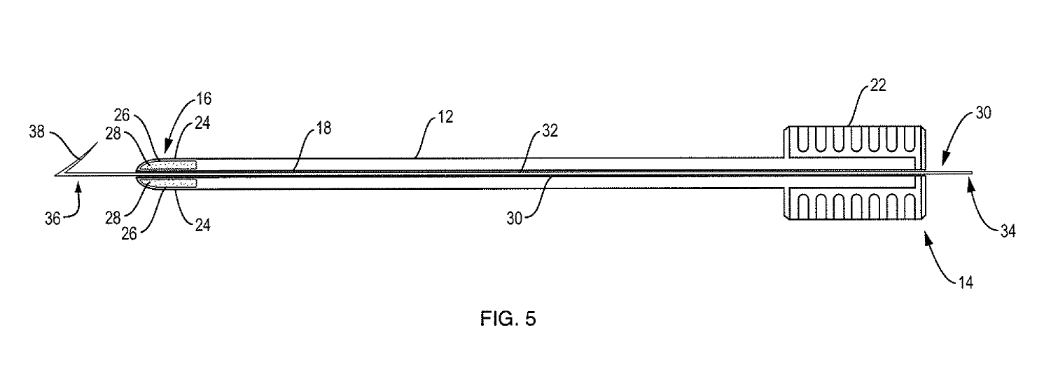

Drawing attention to FIG. 4, an embodiment of a localizing wire 30 is illustrated. The localizing wire 30 includes an elongate body 32 having first and second ends 34, 36. An anchoring member 38 adapted to engage tissue is formed on one of the first and second ends. In the illustrated embodiment, the anchoring member 38 is formed on the second end 36; having this configuration, the first end 34 serves as the proximal end of the localizing wire 30 and the second end 36 serves as the distal end of the localizing wire 30. The anchoring member 38 is typically a hook-like protrusion as illustrated in FIG. 4; however, as recognized by those skilled in the art, the anchoring member 38 can take on any of numerous configurations capable of securing said member to tissue. To allow the physician to perform wire localization under MRI guidance and in conjunction with the tissue localization procedure performed using the obturator 10, the localizing wire 30 is made from one or more MRI compatible materials, so as to not interfere with the performance of the MRI machine. Examples of suitable materials include, but are not limited to, Inconel® 625, titanium, Nitanol or other material with similar magnetic characteristics. Further, as illustrated in FIG. 5, the wire 30 is diametrically sized to pass into the open proximal end 14 of the obturator 10, through the obturator lumen 18, and out the open distal end 16, and its axial rigidity is selected to prevent undesirable bending or coiling during insertion so that the anchoring member will successfully reach the target tissue site of interest.

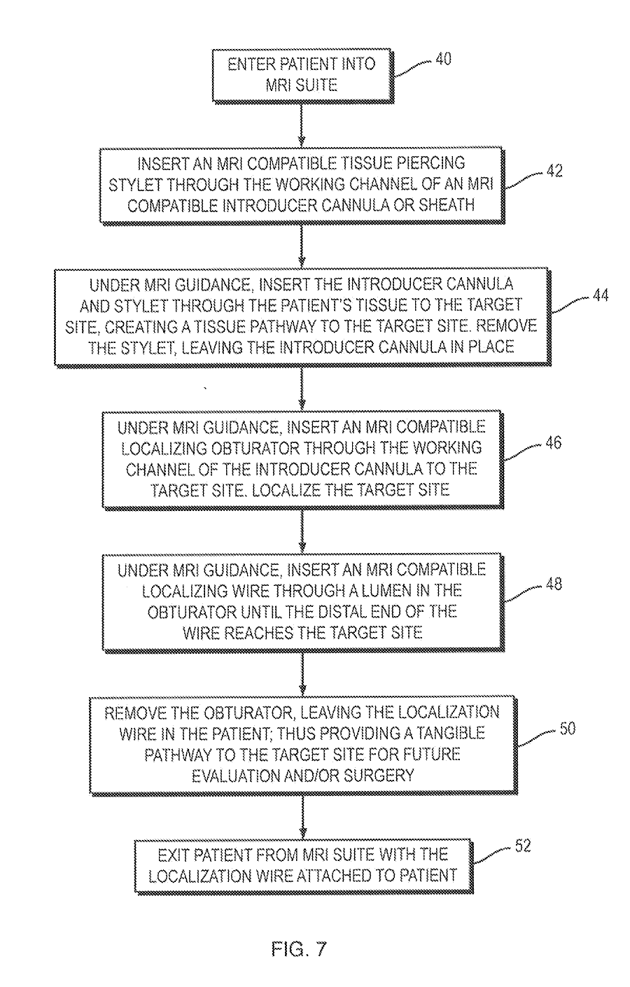

FIG. 7 describes a method of using the localizing obturator 10 in combination with the localizing wire 30 to first localize a target tissue site under MRI guidance for breast cancer screening and/or evaluation, and subsequently perform an MRI guided wire localization procedure. Because the localizing obturator 10 and wire 30 are MRI compatible, both the tissue site localization procedure and wire localization procedure can be performed in the same MRI suite during the same MRI session. The method begins at 40 where a patient is brought into the MRI suite to commence the session. After an initial screening (if performed), at step 42, an MRI compatible tissue-piercing stylet is inserted into the working channel of an MRI compatible introducer cannula or sheath. At step 44, under MRI guidance, the introducer cannula and stylet are depressed and inserted through the Patient's tissue to a target tissue site, creating a tissue pathway to the site. The stylet is then removed leaving the introducer cannula in place relative to the site. At step 46, under MRI guidance, the localizing obturator 10 is inserted through the working channel of the introducer cannula and through the tissue pathway to the target site. Using the obturator, the target site is localized (i.e. the location of the target site is confirmed). Next, at step 48, under MRI guidance, the localizing wire 30 is inserted into the open proximal end 14 of the obturator 10, through the lumen 18 and out of the open distal end 16, until the anchoring member 38 reaches and engages tissue at the target site to secure the wire 30. Moving on to step 50, the obturator 10 is then removed from the patient, leaving the localizing wire 30 in the patient to serve as a tangible pathway leading directly to the target tissue site for future evaluation and/or surgery. With the localizing wire 30 firmly secured to the patient, at step 52, the patient is removed from the MRI suite, ending the treatment session.

Referring now to FIGS. 8-15 a medical procedure using a system 60 employing the target localizing obturator 10 and localizing wire 30 is illustrated. As with the above-described method, the entire procedure can advantageously be performed in a single MRI session (i.e. in a single MRI session without requiring the patient to leave the MRI suite in order to have some of the steps described herein performed; particularly, the tissue localization step using the localizing obturator 10 and the wire localization step using the localizing wire 30 are performed in the same MRI session so that the patient does not have to leave the MRI session and endure a wholly separate wire localization procedure at a later time and date, at a separate location, and under a different imaging modality, which is the current method). Hence the components herein described are MRI compatible where required so as to not interfere with the MRI imaging equipment. The system 60 is used to assist the physician in localizing a target tissue site (denoted generally by mass 80) in a patient's breast 86 for MRI breast cancer screening and/or tissue evaluation, and for performing a subsequent MRI guided wire localization procedure during the same MRI session in advance of a surgical breast biopsy or lumpectomy. A reference structure 82 may be positioned adjacent to the patient to assist in locating the target tissue site 80. The location of the target tissue site 80 relative to the reference structure 82 may be determined along one or more axis. In the illustrated embodiment, the target tissue location relative to reference structure 82 is determined along the X, V and Z axes; however, the target tissue location may also be determined along any combination of said axes.

In one embodiment, the reference structure 82 includes a support grid (not shown) having a number of holes therethrough. Each hole is sized to allow passage of an outer positioning introducer sheath or cannula 70. The hole through which the introducer cannula 70 is ultimately inserted is determined by the location of the target tissue site 80 relative to the reference structure 82 along the X and Y axes. The patient and the reference structure 82 are viewed using a medical imaging system, such as MRI, to determine the location of the target tissue site relative to reference structure 82.

With particular reference to FIGS. 8-9, after application of anesthesia, an introducer stylet 72 having a tissue piercing tip 73 (such as a trocar tip) and a portion of the introducer cannula 70 are inserted through the support 82 and into the patient's breast 86. The introducer cannula and stylet 72 are then advanced through the patient's tissue, thereby creating a pathway 84 to the target tissue site 80. In one embodiment, the introducer cannula 70 is sized to permit only a single instrument therewithin at a time. Thus, the introducer stylet 72 is removed from the patient's body after creating the pathway, leaving behind the introducer cannula 70 (see, e.g. FIG. 9). As an alternative to the introducer stylet, the embodiment of the obturator 10 with a tissue-piercing tip 21, 23 (shown in FIGS. 6A-B) can be employed in the same manner. If the obturator 10 includes an integrated tissue-piercing tip 21 (FIG. 6A), the obturator 10 can remain within the introducer cannula 70 to perform the localization procedure. However, if the obturator 10 includes a selectively removable tissue-piercing tip 23 (FIG. 6B), the obturator 10 can be removed from the introducer cannula 70 if desired to, in turn, remove the tissue-piercing tip 23, leaving the obturator 10 with a blunt tip 20 for reinsertion and target localization.

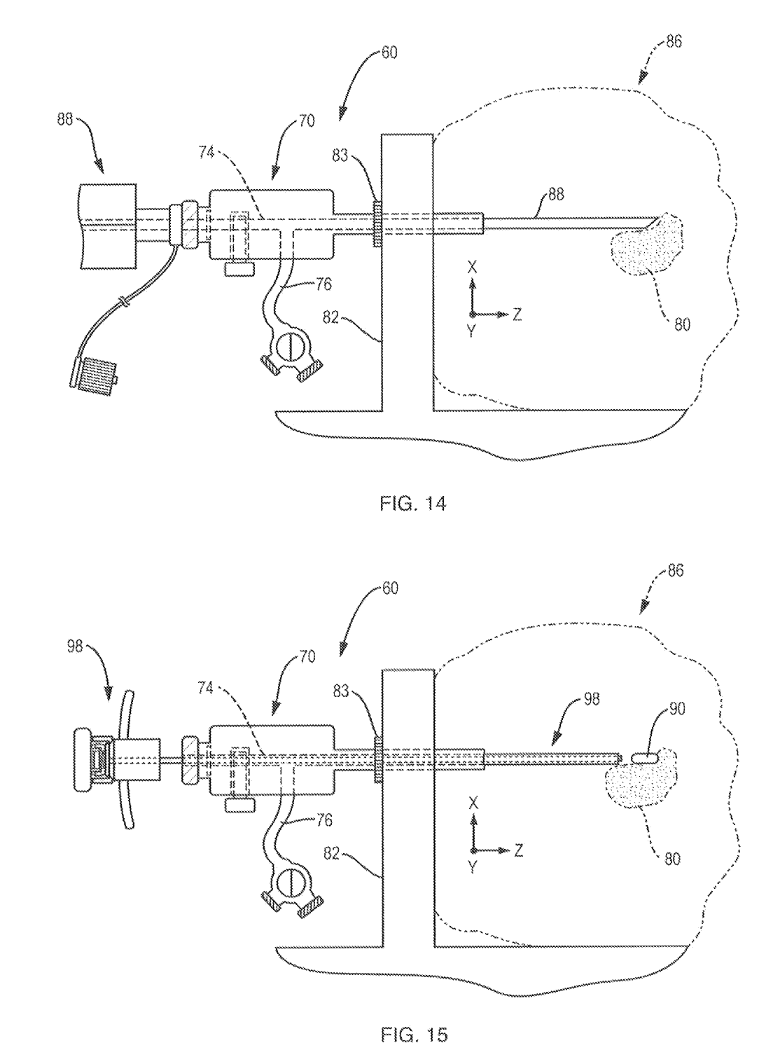

Fluids may be inserted into or removed from the patient's body through an inner lumen 74 in the introducer cannula 70 via a fluid conduit 76. These fluids may include, for example, additional anesthetics and/or saline solution to cleanse pathway 84 and remove blood. Accumulated blood and other fluids within the pathway 84 may be aspirated through the fluid conduit 76 or by inserting an aspirating wand 88 (FIG. 14) prior to insertion of the device 10.

Drawing attention FIG. 10, once the introducer stylet 72 is removed from the introducer cannula 70, a localizing obturator 10 is then inserted into the patient's body through the introducer cannula 70 and tissue pathway created by the stylet 72. With the obturator 10 properly inserted into the introducer cannula 70, the target site 80 is visualized under MRI guidance to determine the location of the site 80 in relation to the reference structure 82. If the target site 80 is in the desired position along the Z-axis, a depth limiting member 83 is moved against reference structure 82 to inhibit movement of introducer cannula 70 and obturator 10 further into the patient, and relative to the target site 80. When no reference structure 82 is used, the depth limiting member may be moved directly against the patient's skin. However, if the target site 80 is not in the desired position, then the position of the obturator 10 and the introducer cannula 70 is modified along the Z-axis until the desired position is localized (i.e. achieved and confirmed) prior to inserting the localizing wire 30.

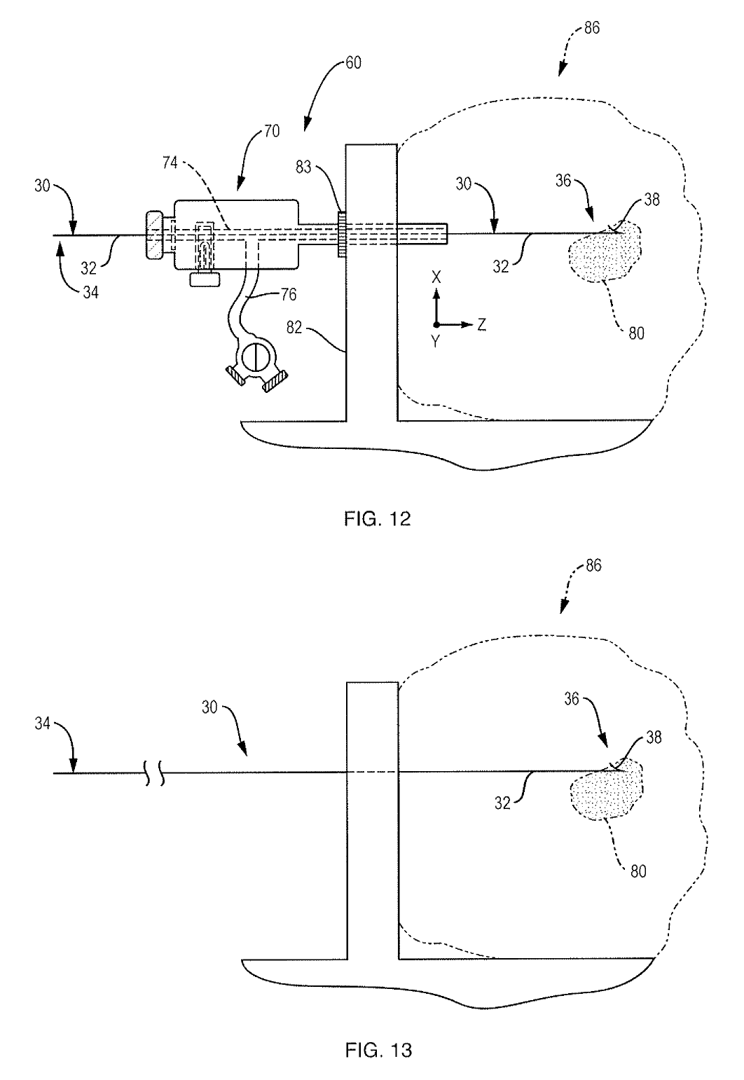

With reference to FIGS. 11-13, with the obturator 10 and cannula 70 in position, a localizing wire 30 is inserted through the lumen 18 of the obturator 10 until the anchoring member 38 engages tissue at the target tissue site 80 to secure the wire 30 (see FIG. 11). With the localizing wire 30 secured at the target site, the obturator 10 is then removed from the introducer cannula 70 (see FIG. 12), leaving behind the localizing wire 30 (see FIG. 13). In this condition, the localization wire is positioned so that its distal end 36 rests at or about the target tissue site 80 and its proximal end 34 extends outside of the patient's breast, thereby creating a tangible pathway capable of leading a surgeon directly to the target tissue site for a future surgical procedure such as a biopsy or lumpectomy.

At any time during the localizing procedures, the target site 80 can be aspirated using an aspirating wand 88 (see, e.g. FIG. 14), or by introducing fluid and/or vacuum through the obturator's optional fluid port 11 and inner lumen 18. Further, if desired, an MRI compatible marker deployment device 98 can be inserted through the introducer cannula 70 as shown in FIG. 15 (with the obturator 10 removed) or through the obturator lumen 18 (with the obturator inserted into the introducer cannula) to deploy a site marker or clip 90 to the target site 80 for future identification. The site marker 90 is preferably imageable under multiple imaging modalities including, but not limited to, x-ray, MRI, tomography, nuclear medicine and/or ultrasound.

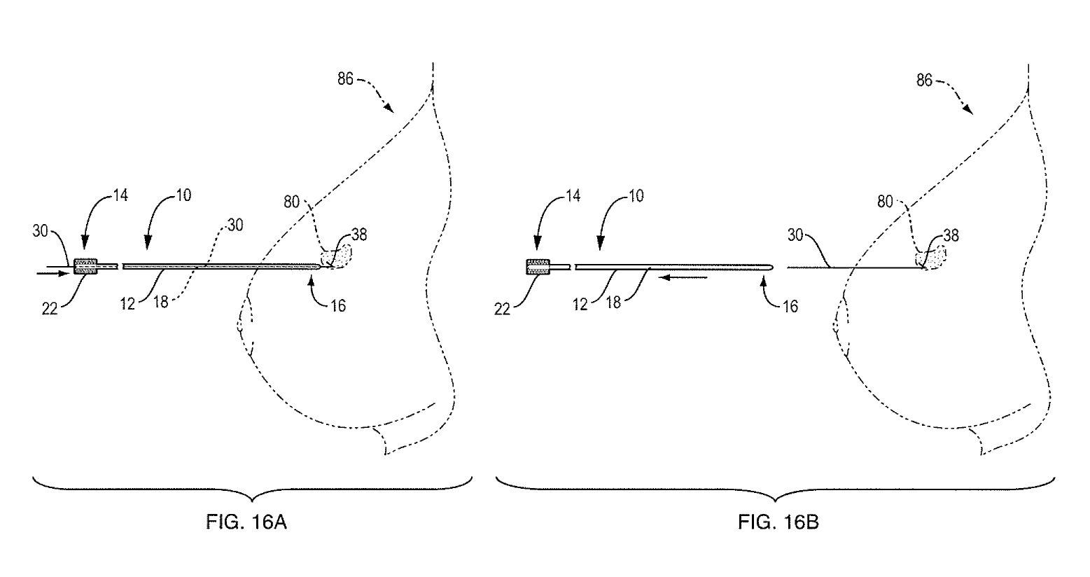

While the above procedure employs a reference structure 82 to locate the target tissue, the reference structure is not necessarily required and a more “free-hand” approach (depicted in part in FIGS. 16A-B) may be utilized to perform the procedure described above (and also the method described in FIG. 7) using the any of the above components including the target localizing obturator 10 and localizing wire 30. For example, as illustrated, the obturator 10 can be inserted by hand into the breast 86 via a pathway created, for example, by an introducer stylet (not shown), as illustrated in FIG. 16A. Once inserted, the breast 86 and obturator 10 are imaged under MRI to localize the target tissue site 80, such as a biopsy site. After the site 80 is localized to determine its precise location, the wire localization procedure is performed by inserting the localizing wire 30 through the obturator lumen 18 until the wire engages tissue at the site 80. Once engaged, and the position of the wire 30 relative to the site 80 is confirmed under MRI guidance, the obturator 10 can be removed as illustrated in FIG. 16B, leaving behind the localization wire 30 as a tangible guiding path for future surgery, evaluation and/or treatment.

The present disclosure has been particularly shown and described with reference to the foregoing embodiments, which are merely illustrative out of the disclosure. It should be understood by those having ordinary skill in the art that various alternatives to the embodiments of the disclosure described herein may be employed in practicing the disclosure without departing from the spirit and scope of the disclosure as defined in the following claims. It is intended that the following claims define the scope of the invention and that the methods, systems and apparatus within the scope of these claims and their equivalents be covered thereby. This description of the disclosure should be understood to include all novel and non-obvious combinations of elements described herein, and claims may be presented in this or a later application to any novel and non-obvious combination of these elements. Moreover, the foregoing embodiments are illustrative, and no single feature or element is essential to all possible combinations that may be claimed in this or a later application.

What is claimed is:

1. A system for localizing a tissue mass, comprising:
an MRI compatible introducer cannula selectively insertable into a patient's tissue, the cannula defining a working channel extending therethrough;
an MRI compatible localizing obturator selectively insertable within the working channel of the introducer cannula and configured for insertion through a tissue pathway leading to a target tissue site, the obturator defining an elongate shaft having an open proximal end, an open a distal end and an inner lumen extending between the open proximal and distal ends;
at least one image enhancing component visible under magnetic resonance imaging (MRI) disposed at least one of on and within at least a portion of the obturator; and
an MRI compatible localizing wire slidable within the inner lumen of the obturator, the localizing wire defining an elongate body having first and second ends and an anchoring member formed on one of the first and second ends, the anchoring member adapted to engage tissue at or adjacent to a target tissue site.

2. The system of claim 1, further comprising an introducer stylet selectively insertable within the working channel of the introducer cannula and defining a tissue piercing tip, the stylet configured to create a pathway to a target tissue site through which at least one of the introducer cannula and obturator is inserted.

3. The system of claim 1, wherein the distal end of the obturator defines a blunt tip or a tissue-piercing tip.

4. The system of claim 1, wherein the localizing obturator and localizing wire are visible under multiple imaging modalities.

5. The system of claim 1, wherein the obturator further comprises a port at its proximal end, the port adapted to connect to at least one of a fluid and vacuum source to introduce at least one of fluid and vacuum through the inner lumen.

6. The system of claim 1, wherein the distal end of the obturator defines at least one of (i) an outer surface including the at least one MRI image enhancing component and (ii) at least one inner chamber including the at least one MRI image enhancing component.

7. The system of claim 1, wherein the image-enhancing component is at least one of a metallic material and a liquid contrast agent.

8. The system of claim 1, further comprising a marker deployment device insertable within the working channel of the introducer cannula and adapted to introduce at least one image marker into a target tissue site.

9. The system of claim 1, further comprising a structure adapted to support and position the introducer cannula relative to a target tissue site.

10. The system of claim 1, further comprising a selectively removable tissue piercing tip attached to the distal end of the obturator.

11. A medical procedure, comprising:
inserting an MRI compatible localizing obturator through a tissue pathway and into a target tissue site, the obturator defining an elongate shaft having an open proximal end, an open distal end and an inner lumen extending between the proximal and distal ends, wherein at least a portion of the obturator is visible by MRI;
positioning the obturator and localizing the target tissue site under MRI guidance;
selectively inserting an MRI compatible localizing wire through the inner lumen of the obturator and into the target tissue site, wherein at least a portion of the localizing wire is visible by MRI;
positioning the localizing wire under MRI guidance so that a portion of the wire is placed at the target tissue site; and
removing the localizing obturator from the target tissue site and tissue pathway, while leaving behind the localizing wire such that a distal portion of the localizing wire remains in contact with the target tissue site and a proximal portion of the wire extends completely through the tissue pathway.

12. The medical procedure of claim 11, wherein the entire procedure is performed in a single MRI session.

13. The procedure of claim 11, wherein the localizing wire defines an elongate body having first and second ends and an anchoring member formed on one of the first and second ends, the anchoring member adapted to engage tissue.

14. The procedure of claim 13, wherein the step of inserting the localizing wire further comprises positioning the anchoring member to engage tissue within or adjacent to the target tissue site to secure the localizing wire to tissue.

15. The procedure of claim 11, wherein the distal end of the obturator defines at least one of (i) an outer surface including at least one MRI image enhancing component and (ii) at least one inner chamber including at least one MRI image enhancing component.

16. The procedure of claim 11, further comprising the step of providing a structure adapted to support and aid in positioning the obturator relative to a target tissue site.

17. A method for localizing a breast biopsy site, comprising:
inserting an introducer stylet through the working channel of an introducer cannula;
inserting the stylet and introducer cannula into a patient's breast tissue, creating a pathway to a target tissue site;
inserting an MRI compatible localizing obturator into the working channel of the introducer cannula and through the tissue pathway to the target site, the obturator defining an elongate shaft having an open proximal end, an open distal end and an inner lumen extending between the proximal and distal ends, wherein at least a portion of the obturator is visible by MRI;
positioning the obturator and localizing the target tissue site under MRI guidance;
selectively inserting an MRI compatible localizing wire through the inner lumen of the obturator and into the target tissue site, the localizing wire defining an elongated body member having first and second ends and an anchoring member formed on one of the first and second ends, the anchoring member adapted to engage tissue;
positioning the localizing wire under MRI guidance so that the anchoring member engages tissue at or adjacent to the target tissue site to maintain the wire in contact with the target tissue site; and
removing the localizing obturator from the patient while leaving behind the localizing wire such that a distal portion of the localizing wire remains in contact with the target tissue site and a proximal portion of the wire extends through the tissue pathway and outside of the patient.

18. The method of claim 17, further comprising the step of providing a structure adapted to support and position the introducer cannula relative to a target tissue site.

19. The method of claim 17, wherein the distal end of the obturator defines at least one of (i) an outer surface including at least one MRI image enhancing component and (ii) at least one inner chamber including at least one MRI image enhancing component.

20. The method of claim 17, further comprising the steps of selectively inserting a marker deployment device into the working channel of the introducer cannula and delivering a site marker to the target site.

21. The method of claim 17, wherein the step of inserting the stylet and introducer cannula into a patient's breast tissue is performed under MRI guidance.

22. The method of claim 17, wherein the entire method is performed in a single MRI session.

23. The method of claim 17, further comprising the step of removing the stylet from the introducer cannula before inserting the localizing obturator.
